(19) United States
(12) Patent Application Publication
(10) Pub. No.: US 2007/0203581 A1
(43) Pub. Date: Aug. 30, 2007

(54) Lumbar disc prosthesis

(76) Inventor: Vicente Vanaclocha Vanaclocha, Valencia (ES)

Correspondence Address: JACOBSON HOLMAN PLLC, 400 SEVENTH STREET N.W., SUITE 600, WASHINGTON, DC, 20004, US

(21) Appl. No.: 11/516,653

(22) Filed: Sep. 7, 2006

(30) Foreign Application Priority Data
Sep. 7, 2005 (ES) 200501990

(51) Int. Cl. A61F 2/44

(52) U.S. Cl. 623/17.14

(57) ABSTRACT

The invention refers to a new type of disc prosthesis for application to the lumbar area especially designed to permit movement within the normal physiological limits of the invertebral disc, permitting lateralisation, flexion and extension under conditions which are as close as possible to the physiological ones.

### OBJECT OF THE INVENTION

The invention refers to a new type of disc prosthesis for application to the lumbar area especially designed to permit movement within the normal physiological limits of the invertebral disc, permitting lateralisation, flexion and extension under conditions which are as close as possible to the physiological ones.

### BACKGROUND OF THE INVENTION

At the present time, one inconvenience of existing lumbar disc prostheses is that they permit excessive movement, i.e., above the physiological line. Excess movement above the physiological line causes regulation of the articulated box in the long term. One movement in particular that is poorly controlled is rotation. That is why our prosthesis permits movement on all planes but always within the physiological limits. In addition, and if the system fails, there is a blocking device that makes it possible through intervention to block all types of movement.

### DESCRIPTION OF THE INVENTION

The lumbar disc prosthesis referred to herein basically comprises two pieces that can be adjoined to one another, both pieces adopting a kidney-shaped peripheral formation, the two pieces offering a bumpy external surface, with drilling of the horizontal and transversal material to form small, sharp pyramidal elevations covering the entire external configuration of both parts of the prosthesis.

The upper piece of the lumbar disc, on the plane on which it makes contact with the lower piece, contains an ample oval-shaped, totally rounded cavity which can house the likewise oval-shaped protruding part of the lower piece to conform an oval-shaped central core with a central belt, thereby limiting all of the movements to the normal physiological limits of the invertebral disc. In particular, it is the only disc that limits rotation to levels within the normal range, also limiting lateralisation, flexion and extension under conditions that are appropriate for the function which the prosthesis is intended to perform, incorporating a blocking system that prevents either piece of the prostheses from moving forward to cause a dislocation.

Another feature of this lumbar disc prosthesis is that is comes with assembly bolts that make it possible to block the disc with a rear mooring, the same one that is used to perform a lumbar arthrodesis.

To join the pieces together, special bolts are used consisting of a smooth cylindrical body with a sharp end, while the head, which is the last part to be assembled and which is fixed to the upper outside part, is threaded on the outside so that when the bolts are housed in an oblique position, they will go through both pieces (the lower one through the oval-shaped domed protrusion) and the upper part which will house the sharp end, threaded finally by their respective heads at the entrance of the screw itself into the sides of the upper part, conforming the fully assembled lumbar disc prosthesis.

### DESCRIPTION OF THE FIGURES

For a better understanding of the general features described above, enclosed herewith are figures which graphically and schematically represent a preferred practical embodiment of the lumbar disc prosthesis of the invention. It should be noted, given the eminently informative nature condition of the drawings in question, that the figures shown therein should be examined with discernment and without limitation, which figures represent the following:

FIG. 1 shows a frontal view of the outside face of the upper part which adopts a kidney shape, the entire surface presenting a bumpiness formed of transversal and longitudinal slots that produce a plurality of small, sharp pyramidal protrusions.

FIG. 2 shows a frontal view of the inside face of the upper piece, with an oval-shaped central cavity and threaded orifices on one edge to house the blocking bolts.

FIG. 3 shows the same frontal view of the inside as FIG. 2, in which the blocking bolts are assembled, positioned in an oblique arrangement, threaded by their heads to the upper part of the prosthesis.

FIG. 4 shows a profile cross-section of the upper part, where the oval-shaped central cavity and the threaded orifices for the introduction of the blocking bolts are seen.

FIG. 5 shows a frontal view of the inside of the kidney-shaped lower piece presenting an oval-shaped central protrusion with a central bolt, with the arrangement of orifices which run obliquely through the oval-shaped protrusion for the blocking bolts carrying the outside face with a serrated edge along the entire periphery.

FIG. 6 shows a longitudinal profile of the lower piece according to FIG. 5, where the oval-shaped protrusion and the orifices for the blocking bolts can be seen.

FIG. 7 shows a longitudinal side view of the fitting system for the two pieces comprising the lumbar disc prosthesis, the outer surfaces of which two pieces are slightly jagged.

FIG. 8 shows an elevation view of one of the bolts.

FIG. 9 shows a longitudinal projection view of one of the blocking bolts, with threading on the outside of the head.

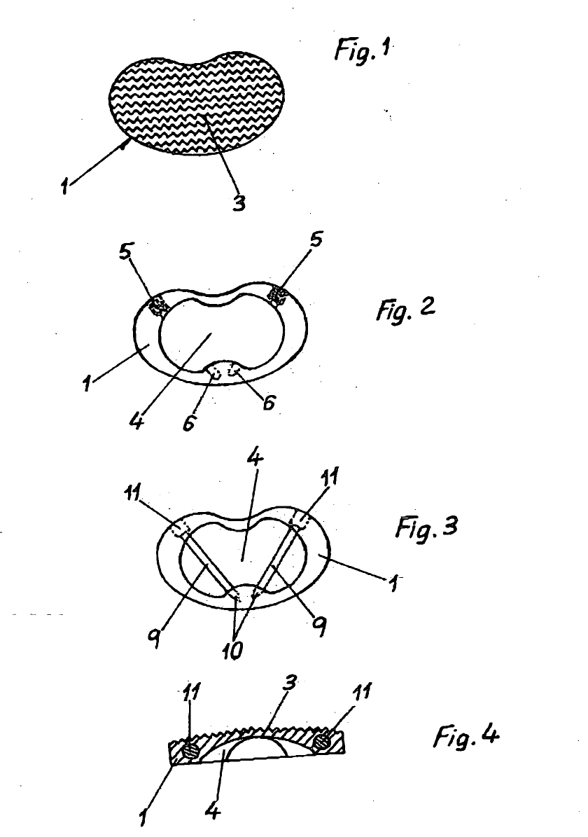

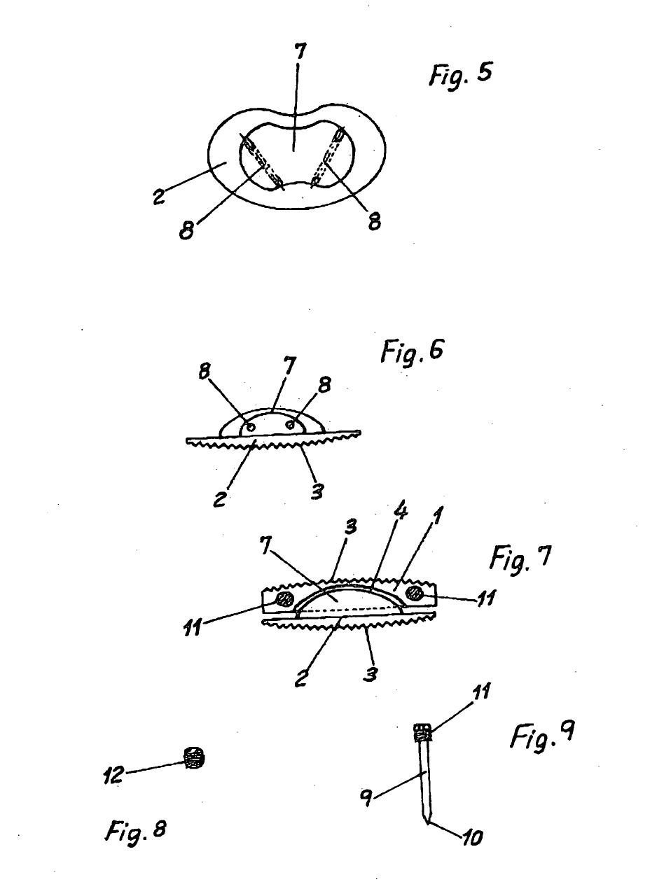

### PREFERENTIAL EMBODIMENT OF THE INVENTION

Always referring to the attached drawings, it should be noted that in the different figures designed therein, numerical elevations have been incorporated related to the descriptions of the characteristics and operations described below, thereby facilitating their immediate location. Hence, (1) and (2) are the complementary upper and lower parts of the disc prosthesis, respectively, the outer surfaces of which parts (1) and (2) are serrated (3) caused by a plurality of longitudinal and transversal slots constituting small elevated pyramids with their sharp edges.

The inside plane of the upper piece (1) has an oval-shaped cavity (4) constituting a union with the lower piece (2), with threaded orifices (5) on the periphery and one of the longitudinal sides followed by a smaller diameter cylindrical prolongation which extends to the oval-shaped central cavity, the aforementioned orifices being position from the edge in an oblique arrangement so that the orifices themselves on the inside wall opposite the oval-shaped cavity (4), are prolonged in the position (6), permitting the assembly and fastening of the lower piece (2).

To enable the assembly of the prosthesis, the internal and central plane of the lower part (2) contains an oval-shaped central protrusion (7) perfectly adaptable to the cavity (4) also oval-shaped, of the upper piece (1), this piece (2) having in its central protrusion two orifices (8) arrangement obliquely and in conjunction with the threaded (5) and blind (6) orifices to permit the assembly of the blocking bolts (9) with the sharp point (10) and the threaded head (11), thereby fitting both pieces (1) and (2) together to form the lumbar prosthesis, the assembly also containing bolt covers (12) to cover the threaded sector (5) of the upper piece (1) of the prosthesis.

Having amply described each and every one of the parts constituting the lumbar disc prosthesis of the invention, all that remains is to note that the different parts can be made from a variety of materials in a variety of shapes and sizes, and the possibility of introducing construction variation, provided that they do not alter the essential aspects of the invention which is the objective of this Utility Model application.

1. Lumbar disc prosthesis characterised in that is composed of two assemblable parts that adopt a peripheral kidney shape, the outer surfaces of which are serrated by transversal and longitudinal slots conforming pyramidal protrusions with sharp edges, the upper piece of the two having an oval-shaped cavity positioned centrally on the inside face and on the longitudinal side of the external hollow in the shape of a kidney from the edge are arranged two threaded orifices followed by a prolongation of a smaller diameter, whose orifices are arranged in an oblique position and in correspondence aligned with blind orifices on the longitudinal side opposite the central cavity, thus allowing the assembly of the two parts of the prosthesis.

2. Lumbar disc prosthesis according to the previous claim characterised in that the lower piece of the two, on its inside, has an oval-shaped central protrusion, adaptable to the central cavity of the upper piece, in whose central protrusion from the lower piece are positioned two pieces obliquely and in correspondence in alignment with the oblique orifices of the upper part, arranging as joining elements some blocking bolts with sharp points and the head threaded on the outside for adaptation to the threaded orifice of the upper piece, going through the assembly bolts the central core of the lower piece, thereby obtaining the fixation of the parts, also having cover bolts adaptable to the threaded orifices of the upper piece.
